(19) United States
(12) Patent Application Publication
(10) Pub. No.: US 2011/0104064 A1
(43) Pub. Date: May 5, 2011

(54) CYCLIZED NON-NATIVE SYNTHETIC PEPTIDE AND COMPLEX OF PEPTIDES COMPRISING SAID CYCLIZED PEPTIDE, INTENDED TO INDUCE AND TO CHARACTERIZE THE PREVENTION OR TREATMENT OF CONDITIONS IN MAMMALS

(75) Inventors: Serge Vicens, Gignac la Nerthe (FR); Grégory Hottin, La Londe les Maures (FR)

(73) Assignee: ORIDAN INC., Claymont, DE (US)

(21) Appl. No.: 12/999,334

(22) PCT Filed: Jun. 18, 2009
(86) PCT No.: PCT/FR2009/000730

§ 371 Date: Dec. 16, 2010

(30) Foreign Application Priority Data
Jun. 19, 2008 (FR) 08/03436

(51) Int. Cl. A61K 39/00; C07K 14/00; C07K 17/02; C07K 17/14; C07K 17/12; C07K 17/08; A61K 49/00; A61K 38/16; G01N 33/53; C12N 1/10; A61P 37/04; A61P 33/02

(52) U.S. Cl. 424/9.1; 530/317; 530/403; 424/185.1; 514/4.4; 435/7.92; 435/258.3

(57) ABSTRACT

Non-native synthetic peptide cyclized by means of a disulphide bridge, characterized by the amino acid sequence (E34P) that follows (SEQ ID No. 1): E-D-E-H-K-G-K-Y-C-R-L-G-N-D-C-R-T-T-E-P-T-T-T-A-T-P-R-G-T-P-T-P-A-P and a complex of non-native synthetic peptides that is intended to induce and characterize the prevention and/or treatment of conditions in mammals, the protective immunity of which depends on the stimulation of Th1 type lymphocytes, and, in particular, on a state of delayed hyperstimulation, containing: —said peptide (E34P), —the following sequence (A16E) of 16 amino acids (SEQ ID No. 2) or derivatives (structural analogues) thereof: A-A-R-C-A-R-C-R-E-G-Y-S-L-T-D-E —the following sequence (A16G) of 16 amino acids (SEQ ID No. 3) or derivatives (structural analogues) thereof: A-A-S-S-T-P-S-P-G-S-G-C-E-V-D-G, —and an adjuvant which preferably induces a cell-mediated response.

The present invention relates to a non-native synthetic peptide cyclized by means of a disulfide bridge and its mixture with two other non-native synthetic peptides as well as an adjuvant, to form a complex intended to induce and to characterize the prevention or the treatment of conditions in mammals the protective immunity of which depends on the stimulation of Th1 type lymphocytes and, in particular, on a state of delayed hyperstimulation.

This complex is more particularly notable in that it enables significant production of specific antibodies of isotype IgG2 to be induced, allowing, in the case of conditions linked to a delayed state of hyperstimulation, the distinction between an infected or sick animal or human and a non-infected or healthy, but vaccinated or treated, animal or human.

Finally, the present invention is related to a reagent inducing stimulation of Th1 type lymphocytes, notably for the prevention or treatment of conditions linked to a state of immediate hyperstimulation and as in vitro diagnostic reagents allowing the detection of vaccination or treatment antibody markers, that is to say, IgG2 specific to the cyclized peptide. Finally, the present invention also relates to the neutralizing role of these IgG2 characterized by the inhibition of the proliferation of promastigote or amastigote forms of Leishmania.

Numerous pathological conditions are linked to a Th1 or Th2 type immune state. For example, the exacerbation of the Th2 pathway corresponding to a state of immediate hyperstimulation induces certain conditions such as canine atopic dermatitis, allergies and asthma.

Curing these conditions is carried out by immunostimulants allowing the transition from a Th2 type to a Th1 type immune state.

On the other hand, this Th1 immune state is in full agreement with a state of resistance to intracellular pathogens such as Leishmania, Trypanosoma, Candida, Mycobacterium and Listeria.

Concerning the immunopathology of asthma, the literature throughout the last decade has designated the allergen-specific T lymphocyte as the main player in the immuno-allergic reaction (Magnan. A et al. “Cytokines, from atopy to asthma: the Th2 dogma revisited” Cell Mol. Biol., 2001, 47, 679-687). Among the T lymphocytes, the Th2 subpopulation producer of IL4 appears to be the focus and necessary for the induction of this reaction. Hence, it is natural that strategies are established for specifically targeting lymphocytes and in particular Th2.

This type of strategy may also be applied to leishmaniases.

Leishmaniases comprise a complex collection of parasitic infections which are rife throughout vast regions of the world (88 countries, spread over 4 out of the 5 continents, are affected) and which are responsible for a large spectrum of clinical conditions (cutaneous, mucocutaneous and visceral, the latter being the most serious form as it is fatal in the absence of treatment).

350 million people are exposed to the risk of contracting them; about 12 million being carriers of the parasite and the annual incidence is estimated at between 2 and 2.5 million new cases, among which 500,000 people are suffering from visceral leishmaniasis.

Visceral leishmaniasis (VL) is caused by parasites of the Leishmania donovani (L. infantumlchagasi and L. donovani) complex, which are intracellular parasites compulsory of the macrophage. In contrast to other species of Leishmania causing cutaneous forms of the disease (L. major, L brasiliensis, etc.), leishmanias of the donovani complex have the ability to spread to all of the internal organs where they multiply. Patients suffering from VL show a clinical picture combining a moderate but persistent fever, hepatosplenomegaly and pancytopeny generally leading to death if no specific treatment is started sufficiently quickly.

Wild dogs and domestic dogs are the main sources of L. chagasi in Central and South America, and of L. infantum in the Mediterranean basin, spreading to certain regions of the Middle East and Asia. In these regions, strongly endemic with canine leishmaniasis, the incidence of the illness in humans is very rare.

Even though VL is a major problem in Southern Europe with the extension of the AIDS pandemic and the appearance of a growing number of Leishmania/HIV co-infections, it is far from being controlled and is becoming very worrying in terms of a public health problem. In other parts of the world, notably in zones endemic with L. donovani, it is estimated that around ⅕ of the population in Sudan and India where fatal epidemics have caused hundreds of thousands of victims over the last few decades. Furthermore, the situation is becoming critical in India with the emergence of strains resistant to pentavalent antimonies (first-line medication).

The control of leishmaniases has necessarily resorted to chemotherapy. This latter treatment is unfortunately jeopardized by long, toxic and costly treatments accompanied with a number of cases of relapse and by the emergence of chemo-resistant phenomenon. It currently seems obvious that the control of this parasitic endemic will depend on the discovery of new prevention and treatment methods that are affordable to the population concerned, with vaccination being the most appropriate method.

The fact of being able to induce an acquired immunity to re-infection in an individual who has been voluntarily infected with living parasites very quickly demonstrated that vaccination against these parasites was possible, at least for the cutaneous forms of leishmaniasis. These practices, still called leishmanisation, used for a long time in the Middle East, Iran and ex-USSR, have been subsequently abandoned due to their harmfulness.

Among the vaccine candidates proposed during the last few decades, chronologically, there were first generation vaccines using live, dead or attenuated promastigotes which, in mice, have shown mixed results. Then, more recently, the advancement of molecular biology techniques enabled the development of sub-unit vaccines or those comprising recombinant proteins such as GP64, GP46, lipophosphoglycan (LPG), etc., mainly the major recombinant proteins exposed to the parasite surface. Some of these recombinant proteins provided a good level of protection against an experimental infection in mice. Finally, third generation vaccines exist, which involve the functional genome and use transgenes such as microorganisms transfected with the gene GP63 and recombinant leishmanias with attenuated virulence or DNA vaccines.

From all of this work, there are three observations:
Vaccination against cutaneous leishmaniasis has been the subject of numerous studies compared to those concerning visceral leishmaniasis. However, the experimental infection models for visceral leishmaniasis are more difficult to carry out (notably because of the difficulty of experimentally infecting rodents).
Some extremely interesting results have been obtained in mice. However, it is difficult and hazardous to transpose the results obtained in mice directly to natural leishmaniasis infection models (dogs, humans). The work of Dunan et al. conducted in 1989 is a very illustrative example of this. These authors had obtained very good protection in mice against a test infection with L. infantum by using an antigenetic fraction called LiF2. This same fraction, injected into dogs, not only did not protect them but, more seriously, increased their susceptibility to the infection.
Few vaccine candidates have passed the experimental stage (mice, hamsters) because of the difficulty of carrying out long, costly and tedious clinical trials.

Therefore, when the studies are not about cutaneous leishmaniasis but about visceral leishmaniasis, the principal studies related to vaccination have still been carried out on mice. However, these experimental infection models are not relevant because they are far-removed from the natural infection hosts, i.e., dogs and humans, hosts having considerable impact in terms of veterinary and human health. Furthermore, as previously indicated, these studies hardly concern anything other than cutaneous leishmaniasis and notably L. major (principal infection model in mice), for which the studies are not transferable to visceral leishmaniasis.

Therefore, the difficulty in reaching the objective of the present invention is, on the one hand, to conduct a study on visceral leishmaniasis, and, on the other hand, to obtain results which are effectively and harmlessly transferable to dogs and humans.

To be useful in a natural host, a vaccine against leishmaniasis must satisfy several general criteria:
Possess a good level of innocuousness (absence of local and general toxicity in the vaccine candidate, absence of toxicity linked to the induced immune response).
Provide an excellent level of protection in a natural host of the infection. All research work focusing on the immunology of leishmaniases agrees that the focusing of immune responses towards a Th1 type pathway is responsible for the state of protection.
Be low cost. Leishmaniases are parasitic diseases termed “neglected” as they affect the populations of the poorest countries. It is therefore important to make new methods of prevention accessible to the populations concerned.
Offer good stability enabling maximum distribution of the vaccine in poor countries where leishmaniasis is principally rife.
To have good industrial feasibility. A synthetic vaccine having all industrial benefits faced with the production of a vaccine requiring the mass culture of parasites.

Two other more specific eligibility criteria must also be taken into account:
The ability that a vaccine possesses to induce immune responses enabling the distinction between a vaccinated host in an endemic zone and an infected host. Indeed, as for numerous parasitic diseases, the clinical diagnosis remains uncertain as the symptoms are not very specific and often absent (numerous asymptomatic carriers exist) and only really appear at a very advanced stage of the infection, which then becomes very difficult to treat. Serological diagnosis which consists of showing the presence of specific antibodies circulating is routinely practiced (indirect immunofluorescence technique). A vaccine, when administered, generally generates specific antibodies which very often can also be produced during an infection. The presence of such antibodies in a host can therefore be confusing in the sense where infection and vaccination possess the same signature.
The advantage of giving a cross-immunoprotection for different leishmaniases. The fact of targeting one or more of the antigen(s) common to all species of leishmanias with vaccine must, without any doubt, represent a real advantage in terms of cross-vaccination.

The present invention remarkably and advantageously meets all of these conditions.

Recently, a vaccine candidate composed of excretion-secretion antigens (ESA) of L. infantum promastigotes formulated with the muramyl dipeptide has been proposed (Patent FR 2825633 and FR 01/07606). A vaccination trial in dogs has enabled a Leishmania infantum in vivo immunity study model to be established in dogs, natural reservoir host of visceral leishmaniasis. They are, in effect, naturally receptive to the L. infantum/chagasi cycle and represent a potential source of parasites which are transmissible to humans. Phase I safety studies in dogs have shown very good local and general tolerance to the vaccine candidate. No toxicity linked to the induced immune response was shown during different safety protocols. The evaluation of its efficacy showed that it gave 100% protection to the test infection (phase II, Lemesre et al. Vaccine 23, 2005, 2825-2842) and, notably, that it protected dogs subjected to natural infections for two transmission seasons in an endemic zone (study carried out on 414 dogs recruited by 18 clinical veterinaries) with a percentage efficacy close to 90% (phase III, Lemesre et al, Vaccine 25, 2007, 4223-4234).

Vaccination by ESA controls not only the development of the disease but also significantly decreases the parasitic burden of the dog and thus contributes to the interruption of the transmission of leishmanias.

All of the results firmly reinforce the fact that the vaccine candidate (combination of ESA promastigotes of L. infantum with the adjuvant MDP) protects dogs against visceral leishmaniasis from L. infantum by durably inducing a polarization of the cell-mediated response towards a Th1 type profile leading to the production of IFN-g, capable of activating the macrophage and of inducing the production of nitrous oxide (NO) causing the destruction of the intracellular amastigotes by apoptosis (patents FR 2825633, FR01/07606 and FR 02/09506).

Alongside the immunoprophylactic tests, immunotherapeutic tests have given promising results (Patent FR 02/09506). Vaccinated dogs induced a specific and durable antibody response of isotype IgG2, at low levels and mainly directed against a major immunogene of 54 kDa compared to a placebo group. The major immunogene of ESA has been identified by genetic engineering as belonging to the “promastigote surface antigen” family, known as PSA.

These latter are characterized by the presence of a repeat unit rich in leucine (Leucine Rich Repeat, LRR) and a carboxyterminal group rich in serine/threonine residues.

The document of Lohman/Mc Mahon Pratt (1990) PNAS 87: 8393-8397, is known, which reports the cloning and sequencing of the gene encoding for the native protein GP46/M2 in the promastigote form of Leishmania amazonensis, and notes its implication in the immunization against leishmaniases, notably the cutaneous form, in experimental murine models.

This document notes that the native protein GP46 maintains a tertiary or quaternary spatial conformation and is an immunoprotector in mice in a cutaneous leishmaniasis model. This observation naturally leads the researcher to think that this particular conformation is involved in the immunodominant character of the protein. Moreover, research has been carried out in this direction, further to said document: Sjölander et al. (1998) PH: S0264-410X/98. This study showed that the protein GP46, produced in the recombinant form in a bacterial system (E. coli), regardless of the structures, is no longer protective and loses its efficacy.

This, logically, leads one to think that the secondary, tertiary or quaternary spatial conformation of the protein GP46 is important in the protective efficacy of said protein. However, faced with these publications, the inventors of the present application have continued their work on synthetic peptides, which because of their size and their preparation method can only bring benefits.

The interest in such peptides is clearly multiple: avoidance of problems linked to the isolation of peptide sequences, reducing the cost and the time required to carry out this operation therefore increasing productivity with an obvious financial gain, enabling the risk of infection to be brought under control. Such peptides meet, in a remarkable way, all of the conditions previously cited: stable, low cost and effective vaccine, suitable for poor countries where leishmaniasis is principally rife.

Although PSA (PSA-2) had been initially identified in promastigote forms, different studies seem to indicate that it is also present in amastigote forms. Furthermore, it has recently been shown that this PSA was even excreted-secreted by the two principal parasitic stages and showed remarkable immunological properties in the sense where dogs immunized with promastigote ESA from L. infantum were capable of inducing a specific antibody response of isotype IgG2 specifically directed against the excreted-secreted PSA (Patent FR 02/09506).

In Leishmania no functional biological role has been attributed to them whereas in other pathogens such as bacteria, LRR proteins such as InIB, Yop and Ssph are involved in the virulence and the pathogenicity of these organisms (Marino et al., 2000, PNAS. 97(16):8784-8788).

A noteworthy fact is that in plants, they are involved in the resistance to different pathogens. Very recently, studies on the involvement of PSA (Promastigote Surface Antigen) of leishmanias at the cellular adhesion level (Kedzierski et al., 2004, J. Immunol., 172; 4902-6), of the resistance of parasites to lysis by the complement (Lincoln et al., Mol. Biochem. Parasitol. 2004, 137:185-9) and its differential expression during the leishmanias life cycle (Handman et al., 1995, Inf. Immun., 63: 189-200) have tended to show that the PSA constitutes a vital and essential component in the development of leishmanias.

Native PSA-2 from L. amazonensis gives a good level of protection in mice against an experimental infection of L. major by inducing a Th1 type cell-mediated protective immunity (Handman et al., 1995, Infect. Immun. 63: 4261-4267). In contrast, a recombinant PSA protein produced in a bacterial expression system does not give this protection, leading to the suggestion that the post-translational modifications of the protein constitute an indispensable stage in its protective activity (MacMahon Pratt, 1996).

More recently, in dogs, the use of a couple of peptides (in the form of lipopeptides) of 16 amino acids, each give immunoprotection against a test infection, similar to that developed during immunization with ESA. Dogs vaccinated in this way have a cell-mediated response towards Leishmania infantum (Patent FR 01/11942). 01/11942).

In contrast, this peptide vaccine formed from 2 peptides is incapable of generating a significant specific IgG2 antibody response enabling the distinction, in an endemic zone, between a vaccinated host and a non-vaccinated but infected host.

The present invention is able to provide an advantageous solution to this major difficulty notably by using a non-native synthetic peptide cyclized by means of a disulfide bridge.

This peptide may be used alone in a vaccination or in combination with other peptides.

However, according to a preferred embodiment of the invention, it is used in combination with two non-native synthetic peptides described in patent FRO1/11942 and an adjuvant preferably inducing a cell-mediated response, which provides the optimal vaccination results. The results of the conducted vaccination tests will be discussed later.

This mixture of three peptides and an adjuvant form an inventive complex enabling the disadvantages and inadequacies of previously proposed models to be effectively overcome.

It is to be noted that the cyclized form of said peptide is crucial to the efficacy of the complex according to the invention. Studies carried out by the inventors have indeed been able to establish that the non-cyclized form of this peptide only induces very partial Th1 stimulation and clearly has insufficient efficacy, whether the peptide is used alone or in combination with other peptides. The inventors therefore carried out studies in an attempt to make up for this shortfall and thus discovered that the cyclized form of said peptide described below provided the required solution.

According to the invention, the peptide cyclized by means of a disulfide bridge is composed of a sequence of 34 amino acids and is referred to as: E34P.

This peptide of 34 amino acids (E34P) is composed of the following sequence

(SEQ ID No. 1):
E-D-E-H-K-G-K-Y-C-R-L-G-N-D-C-R-T-T-E-P-T-T-T-A-
T-P-R-G-T-P-T-P-A-P

This peptide is cyclized by means of a disulfide bridge between the cysteine at position 9 and cysteine at position 15.

Peptide E34P is advantageously capable of inducing a significant antibody response enabling a vaccinated host to be distinguished from a non-vaccinated but infected host in an endemic zone. This cyclization of the peptide is essential for the appearance of specific IgG2 antibodies; cyclization by means of a disulfide bridge between cysteines 9 and 15 causing a specific conformation responsible for this IgG2 synthesis. Indeed, non-cyclization of the peptide at Cys9/Cys15 only induces slight IgG2 synthesis, sometimes difficult to detect.

According to the preferred embodiment of the invention, peptide E34P according to the invention is mixed with a second and third non-native synthetic peptide, each composed of a sequence of 16 amino acids, respectively referred to as: A16E and A16G, or derivatives thereof, as well as an adjuvant.

“Derivatives of sequences A16E or A16G” means all structural analogues of the constituent amino acids of said sequences, known and considered as such by a person skilled in the art.

The peptide of 16 amino acids A16E is composed of the following sequence

(SEQ ID No. 2):
A-A-R-C-A-R-C-R-E-G-Y-S-L-T-D-E

in which C may be replaced by S and L by I.

The peptide of 16 amino acids A16G is composed of the following sequence

(SEQ ID No. 3):
A-A-S-S-T-P-S-P-G-S-G-C-E-V-D-G

in which C may be replaced by S and L by I.

These two latter peptides are capable of inducing stimulation of Th1 type T lymphocytes ensuring protection against visceral leishmaniasis.

Associated with an adjuvant preferably inducing a cell-mediated response, one such triplet of peptides presents all the advantages previously cited (no culture, protection in a natural host of the infection, vaccination marker, cross-immunity, affordable to the populations concerned, etc.). The group of peptides E34P, A16E, A16G and an adjuvant form the peptide complex according to the invention.

The adjuvant associated with these peptide sequences is preferably QA21 saponin, from quil-A or any other derivative thereof, known to those skilled in the art, such as QS21.

In a preferred embodiment, the peptide sequences E34P, A16E and A16G are associated in a ratio E34P/A16E/A16G:10/25/25.

The peptide complex according to the invention advantageously induces significant production of IgG2 isotype antibodies specific to peptide E34P, which, in a remarkable way, enables vaccinated individuals to be distinguished from non-vaccinated, but infected, individuals.

The present invention furthermore relates to the use of the three peptide sequences E34P, A16E and A16G as reagents inducing a stimulation of Th1-type T lymphocytes, notably for the prevention and treatment of conditions linked to a state of immediate hyperstimulation and as in vitro diagnostic agents allowing the detection of vaccination antibody markers, that is to say the specific IgG2 of the peptide fragments. The present invention corresponds to a 2nd generation synthetic vaccine for dogs (the 1st generation canine vaccine corresponds to the development of an alternative excretion-secretion antigen system from Leishmania infantum promastigotes, previously cited) adaptable for a human vaccination formula.

The peptide sequences according to the invention E34P, A16E and A16G may be made immunogenic by bonding to carriers or any other derivative (large KLH type molecules, palmitoyl-type lipopeptides or derivatives) and are administered to candidates in the presence of an adjuvant, and preferably QA21. In a preferred embodiment, the peptide/adjuvant ratio is between 3/1 and 3/3.

In the case of an infection by leishmanias, the studies carried out on dogs have allowed the determination of the optimal vaccinal dose as being 20 μg of QA21 for 60 μg of peptides injected. However, the start of a response was observed from 55 μg of peptides injected.

The reaction mechanism of the peptide complex obtained according to the invention was verified with the aid of specific methods demonstrating that the innovative peptide complex reacts either by Th1-type immunostimulation of the lymphocytic system with the production of IgG2, or by immunomodulation of type Th2 towards type Th1.

Secondly, the neutralizing role of the specific IgG2 is characterized by the inhibition of the proliferation of amastigote or promastigote forms of Leishmania.

This study of the Th1 type immune state was carried out on dogs, after vaccination and also on dogs with leishmaniasis after treatment with this peptide complex.

Infection Test Method:

The infection test consisted of intravenously injecting 10⁸ complement-treated promastigotes in metacyclic phase from a healthy dog and 5×10⁸ peritoneous macrophages from a healthy dog, infected in vitro by amastigotes.

The promastigotes and the infected macrophages were diluted in sterile physiological serum to a final volume of 1.5 mL. This mixture was made up just prior to injection.

A parasitological examination was carried out from samples taken directly from the dog.

A smear from the bone-marrow puncture was made on a slide. This smear, once fixed in methanol was May Grümwald Giemsa stained and studied under an immersion microscope (×1000).

Some bone-marrow samples were cultivated in a biphasic culture medium NNN (Novy and Mac Neal, 1904, 4. Infec. Dis., 1:1-30) of which RPMI 1640 added to 20% of decomplemented fetal veal serum made up the liquid phase. Random subculturing was carried out every 4 to 6 days. The cultures were regularly studied by photon microscope (×400) for 20 minutes.

The parasitemias were quantified as follows:
+/−: elongated, immobile, refringent forms
+: 1 to 5 mobile/field promastigote forms
++: >5 mobile/field promastigote forms
+++: cultivated to confluence.

Evidence of Cell-Mediated Th1 Type Immunity

This evidence was gathered using two techniques:
Assaying the leishmanicidal activity of the monocytes according to the method described previously (Patent FRO1076060);
Detection of dog IgG2, specific to the cyclized peptide E34P and the carboxyterminal group of PSA.

This detection was carried out by the ELISA method according to the microtitration technique of Kweider et al. (J. Immunol, 1987, 138, 299) by using an anti IgG2 conjugate. For this method, the peptide is biotinylated before coating onto microplates. This bonding to large biotin-type molecules has the effect of rendering the cyclized peptide E34P more antigenic. We also use the recombinant protein coding for the carboxyterminal group of the PSA as antigen.

Alongside this study of the Th1 type state, serological monitoring by standard immunofluorescence using slides coated with promastigotes (serological reference method for canine leishmaniasis) was carried out.

The detection of isotype IgG2 antibodies specific to the cyclized peptide E34P and the carboxyterminal group of PSA notably enables the immune response in the vaccinated or treated mammals to be monitored, and therefore the infected or sick mammals to be distinguished from the non-infected or healthy, but vaccinated or treated, mammals, and the efficacy of a vaccination or chemotherapeutic and/or immunotherapeutic treatment to be monitored.

The cyclized peptide E34P or the synthetic peptide complex according to the invention may be used in the manufacturing of a medicine, a vaccine, or an in vitro or an in vivo diagnostic agent, for the induction of the production of IgG2 isotype specific antibodies of the cyclized peptide enabling an infected or sick mammal to be distinguished from a non-infected or healthy but vaccinated or treated mammal, and for the induction or diagnosis in mammals of specific antibodies and more particularly of IgG2 isotype, related to a Th1 type immune state.

The cyclized peptide E34P or the synthetic peptide complex according to the invention may be packaged in a form such that it may be administered via different routes: subcutaneous, intradermal, intramuscular, intravenous, parenteral, and oral.

It may also be used as an in vitro diagnostic product and may be included in a diagnostic kit. In such a case, the in vitro diagnostic product is characterized in that it contains the cyclized peptide E34P or the peptide complex according to the invention, for the detection of a humoral response and particularly of IgG2, notably allowing the detection of a Th1 type immune state and of a state of delayed hyperstimulation, the monitoring of the immune response in vaccinated or non-vaccinated but treated mammals, and the monitoring of the efficacy of a vaccine or of chemotherapeutic and/or immunotherapeutic treatment.

The innovative character of the peptide complex according to the invention not only resides in the induction of a Th1 type cellular response, but also in the production of IgG2 type antibody levels. These IgG2 are detectable by various in vitro methods, for example ELISA, DOT, BLOT, WESTERN BLOT, IMMUNOCHROMATOGRAPHY, LATEX FLUOROPHORES, and any other in vitro method involving a conjugated system or other Ag-Ac visualization systems.

In a diagnostic kit, peptides E34P, A16E or A16G may be bound to a solid support, and for example to nitrocellulose or other polymer membranes, latex and various plastic (polymer) material supports.

Evidence of the Neutralizing Role of the Specific IgG2:

Inhibition of the growth of promastigotes and amastigotes in the presence of IgG2 (Patent FR0209506).

The cyclized non-native synthetic peptide E34P induces IgG2 immunoglobulins capable of neutralizing the proliferation of Leishmania amastigotes and promastigotes in vitro.

Immunotherapy Results

According to specialists such as PINELLI (PINELLI, E et al, Infect Immun, 1994, 62. 229-235) dogs with leishmaniasis corresponding to the activation of the Th2 type lymphocytic system show a heightened antibody response.

This increased production of antibodies corresponds to hyperproteinemia and induces the appearance of immune complexes leading to renal failure (increase of creatinine and sanguine urea).

We have therefore tried to modulate towards a Th1 state by administering intradermally doses of the peptide compound to dogs with leishmaniasis. Monitoring of the immune state, as well as clinical observations, was carried out before and after the treatment.

### EXAMPLE 1

### Dog SENIOR

A Weimar Pointer dog, aged 6 years, belonging to Mr. G showed numerous cutaneous lesions accompanied by a state of general fatigue and a thin appearance, all pointing towards canine leishmaniasis.

The veterinary surgeon, Dr. GH diagnosed leishmaniasis. This diagnosis was confirmed by direct observation under the microscope of leishmanias from a cutaneous layer and serological analysis which gave a positive Leishmania content by immunofluorescence of 1/1600.

Analysis of the immune state before any injection confirmed that the dog was in a Th2 type immune state with heightened antibody content as well as a negative leishmanicidal test. We introduced immunotherapy which consisted of carrying out 3 intradermal injections of 60 μg of peptides (25 μg A16E, 25 μg A16G, 10 μg E34P) and 20 μg QA21, each injection being 3 weeks apart.

One week after the third injection, the dog SENIOR regained his appetite and certain vitality. Dr. GH began to observe slight cutaneous improvement.

One month after the last injection, SENIOR regained a normal clinical appearance with, in particular, an increase in weight of 1 kg and the disappearance of 70% of all skin lesions. Analysis of the immune state confirmed a low anti-leishmania antibody content which fell to 1/400 by immunofluorescence. Equally, the leishmanicidal effect was positive. Furthermore, after treatment, the dog SENIOR showed specific IgG2 for the peptide E34P and the carboxyterminal group of PSA measured by ELISA. These specific IgG2 were absent before any treatment.

A study of parasites in NNN culture medium proved to be negative. 8 months after the treatment, the dog SENIOR showed no change. Biological analyses gave confirmation that SENIOR was still in a Th1 immune state.

### EXAMPLE 2

### Dog THEO

A male Breton Spaniel dog, THEO, aged 5 years, belonging to Mr. K showed clinical signs specific to leishmaniasis. According to Dr. DM, the presence of numerous shiny scales, hair loss around the right eye, ulcerous lesions on the right hind leg joint and a pronounced state of fatigue with notably the appearance of an aged dog. Biological analysis notably with a positive leishmaniasis serology by immunofluorescence of 1/800 reinforced the clinical diagnosis.

Immunotherapy was introduced consisting of 3 intradermal injections of 60 μg of peptide vaccine compound (25 μg A16E, 25 μg A16G, 10 μg E34P) and 20 μg QA21, each injection being 3 weeks apart. Analysis of the immune state before any injection showed that the dog THEO had developed a Th2 type immune system with a strongly positive parasitology from the bone-marrow.

One month after the last injection, the leishmanial clinical signs in THEO were receding with notably the healing of the ulcerous lesions, significant loss of the scales as well as the hair loss around the eye being almost non-existent. Serology showed a fall to 1/400 by immunofluorescence. In contrast, analysis of the cellular response gave confirmation that THEO showed an active Th1 state with quite a significant leishmanicidal effect.

Equally, parasitology was negative (bone-marrow culture in NNN medium).

On a humoral level, the dog THEO showed IgG2 specific to the cyclized peptide E34P and the carboxyterminal group of PSA after treatment, IgG2 measured by ELISA.

The present invention therefore consists of a therapeutic peptide compound which induces the change from a Th2 type immune state with a significant production of antibodies which intensify the clinical manifestations towards a Th1 type immune state leading to recovery and accompanied by specific IgG2 notably of the cyclized E34P.

Peptide Complex Vaccination Results:

The peptide complex was tested on 14 perfectly healthy dogs divided into 7 groups, showing negative leishmaniasis serology, negative parasitology, an absence of cellular response to Leishmania and an absence of IgG2 specific to cyclized peptide E34P. In setting up the groups of dogs, it was planned to compare the peptide complex with the cyclized peptide E34P of the peptide complex with the NON-cyclized E34 complex and to compare the cyclized E34P alone with the non-cyclized E34P alone.

The 14 dogs came from northern France (Auxerre), a non-endemic zone, and were repatriated to south-east France, four weeks before the vaccination during a hibernal period (a period not showing leishmania phlebotome vectors). Among the 7 groups, 2 groups of dogs received the E34P peptide (1 group with cyclized E34P and 1 group with NON-cyclized E34P), 3 groups of dogs received the peptide mixture (A16E, A16G and cyclized E34P) with notably 3 different concentrations of the cyclized peptide E34P. Another group of dogs received the peptide mixture (A16E, A16G and NON-cyclized E34P).

Control group (placebos)
Dog No. 1: Negative control: 3 year old male Beagle
Dog No. 8: Negative control: 3 year old male Beagle

Group A: dogs vaccinated with the 3 peptides and 1 adjuvant at the following concentrations:
A16E: 25 μg
A16G: 25 μg
Cyclized E34P: 25 μg
Adjuvant QA21: 20 μg
Dog No. 2: 8 year old female Beagle
Dog No. 7: 7 year old female Beagle

Group B: dogs vaccinated with the 3 peptides and 1 adjuvant at the following concentrations:
A6E: 25 μg
A16G: 25 μg
Cyclized E34P: 10 μg
Adjuvant QA21: 20 μg
Dog No. 3: 7 year old female Beagle
Dog No. 6: 7 year old female Beagle

Group C: dogs vaccinated with the 3 peptides and 1 adjuvant at the following concentrations:
A16E: 25 μg
A16G: 25 μg
Cyclized E34P: 5 μg
Adjuvant QA21: 20 μg:
Dog No. 4: 9 year old female Beagle
Dog No. 5: 9 year old female Beagle

Group D: dogs vaccinated with the 3 peptides and 1 adjuvant at the following concentrations:
A16E: 25 μg
A16G: 25 μg
E34P non-cyclized by disulfide bridge: 10 μg
Adjuvant QA21: 20 μg
Dog No. 9
Dog No. 10

Group E: dogs vaccinated with 1 peptide cyclized E34P and 1 adjuvant at the following concentrations:
Cyclized E34P: 10 μg
Adjuvant QA21: 20 μg
Dog No. 11
Dog No. 12

Group F: dogs vaccinated with 1 peptide non-cyclized E34P and 1 adjuvant at the following concentrations:
Non-cyclized E34P: 10 μg
Adjuvant QA21: 20 μg
Dog No. 13
Dog No. 14

The vaccine injection scheme was as follows (wk=week):

| D0 | D30 | D60 | D100 |
|---|---|---|---|
| 1st injection | 4 wk- 2nd injection | 4 wk- 3rd injection | 7 wk test |
| | | | infection |
| intra-dermal | intra-dermal | intra-dermal | |

Clinical monitoring of the 14 dogs was carried out every 2 weeks.

Biological analyses were carried out:
Before any injection
1 month after the 3rd injection.

Biological analysis consisted of:
Serology of specific IgG2 of cyclized peptide E34P and of the carboxyterminal group of PSA;
Leishmaniasis serology: quantitative immunofluorescence of the total IgG (sign of infection);
Leishmanicidal activity of the monocytes (cell-mediated response).

It was necessary to add to these analyses the study of Leishmania by direct observation under microscope and culture in NNN medium from the bone-marrow after the infection test.

This study of Leishmania was carried out 4 months after the infection test.

No significant clinical manifestation appeared during the entire study.

Before any injection, the 14 dogs showed negative total IgG serology and parasitology.

The role of the IgG2 on the viability and growth of Leishmania promastigotes and amastigotes was analyzed with immunoserums from dogs vaccinated with the mixture including the cyclized peptide E34P and the serums from the placebo dogs.

TABLE I
Immune state of dogs after vaccination

| | Biological parameters | | | | | | | | |
|---|---|---|---|---|---|---|---|---|---|
| | | | | | | Cellular response, | | | |
| | | | | | | Leishmanicidal | | | |
| | | ELISA serology | | ELISA serology IgG2 | | activity of | | Total IgG by | |
| | | IgG2 vs. PSA Cter | | vs. cyclised E34P | | monocytes | | IFAT | |
| | | | 1 month | | 1 month | | 1 month | | 1 month |
| | | | after the | | after the | | after the | | after the |
| | | Before | 3^{rd} | Before | 3^{rd} | Before | 3^{rd} | Before | 3^{rd} |
| Dogs | | vaccination | injection | vaccination | injection | vaccination | injection | vaccination | injection |
| Placebo | 1 | 0.049 | 0.088 | 0.185 | 0.113 | <0% | 19.9% | — | — |
| Group | 8 | 0.062 | 0.072 | 0.120 | 0.170 | <0% | 10% | — | — |
| Group A | 2 | 0.052 | 0.062 | 0.128 | 0.350 | <10% | 71.5% | — | — |
| | 7 | 0.067 | 0.350 | 0.171 | 1.949 | <0% | 76.6% | — | — |
| Group B | 3 | 0.049 | 0.275 | 0.220 | 0.938 | <10% | 81.4% | — | — |
| | 6 | 0.044 | 0.595 | 0.182 | 1.125 | <0% | 91.5% | — | — |
| Group C | 4 | 0.056 | 0.069 | 0.186 | 0.269 | <0% | 70.3% | — | — |
| | 5 | 0.058 | 0.059 | 0.118 | 0.444 | <0% | 82.2% | — | — |
| Group D | 9 | 0.040 | 0.100* | 0.110 | 0.224 | <0% | 84.5% | — | — |
| | 10 | 0.038 | 0.150 | 0.192 | 0.287* | <0% | 90.1% | — | — |
| Group E | 11 | 0.031 | 0.080 | 0.162 | 0.250 | <0% | 30.5% | — | — |
| | 12 | 0.058 | 0.065 | 0.175 | 0.399 | <0% | 52.7% | — | — |
| Group F | 13 | 0.041 | 0.073 | 0.182 | 0.125 | <0% | 31% | — | — |
| | 14 | 0.045 | 0.058 | 0.129 | 0.170 | <0% | 28.7% | — | — |

ELISA IGg2: Cut-off=0.110 for PSA Cter
   Cut-off=0.290 for the cyclized peptide E34P (Optical Density)
Leishmanicidal activity of monocytes: expressed as percentage inhibition of the parasitic index (Patent FR 01/07606): Cut-off=40%
Total IgG IFAT cut-off≧1/100 (indirect immunofluorescence)
   Bold numbers=positive results
   Stared numbers=slightly positive results

According to Table I, the complex with 3 peptides with notably 10 μg of cyclized peptide E34P (group B: dogs 3 and 6) induces Th1-type cellular immunity with induction of IgG2 isotype specific antibodies. The cyclized peptide E34P alone causes only partial response. The NON-cyclized peptide E34P is less effective than the cyclized E34P.

TABLE II
Total IgG serology and parasitemias carried
out 4 months after the infection test

| | | | Parasitology | |
|---|---|---|---|---|
| | | Total IgG | (from marrow punctures) | |
| | | serology | | Culture in NNN |
| Dogs | | (quantitative IF) | Direct exam | medium |
| Placebo | 1 | 1/100 | − | ++ |
| | 8 | 1/100 | + | ++ |
| Group A | 2 | | − | − |
| | 7 | | − | − |
| Group B | 3 | | − | − |
| | 6 | | − | − |
| Group C | 4 | | − | − |
| | 5 | | − | − |
| Group D | 9 | 1/100 | − | +/− |
| | 10 | | − | − |
| Group E | 11 | 1/200 | − | +/− |
| | 12 | | − | − |
| Group F | 13 | 1/100 | + | + |
| | 14 | 1/100 | − | +/− |

Key: IF=Immunofluorescence (considered positive if the level is ≧1/100)
Parasitology=culture in NNN medium
   −=absence
   +/−=rare, elongated, immobile refringent forms,
   +=1 to 5 mobile/field promastigote forms,
   ++=more than 5 mobile/field promastigote forms,
   +++=cultivated to confluence

According to table II, the complex with 3 peptides with the cyclized peptide E34P and adjuvant induces a Th1 protector type immune state: absence of parasites and total IgG four months after the infection test, in particular for dogs 3 and 6 from group B (dogs immunized with 10 μg E34P, 25 μg A16E and 25 μG A16G).

On the one hand, the cyclized peptide E34P alone is insufficient to induce an effective response. On the other hand, the results from table II correlate with those of table I with regards to the difference between peptides E34P and NON-cyclized E34P: the cyclized structure of peptide E34P seems to be essential.

TABLE III
Percentage inhibition of the parasitic growth
by serums from placebo and vaccinated dogs
(dilution ¼) taken on the 5th day of culture

| | Dogs | | |
|---|---|---|---|
| Response different | 1 | 3 | 6 |
| times | (Placebo group) | (Group B) | (Group B) |
| Before immunisation | 3.4% | 5.8% | <0 |
| After immunisation | 4.4% | 73.4% | 84.3% |

The results obtained show that the IgG2 response of the dogs vaccinated with the 3-peptide formulation with the cyclized peptide E34P not only accompany the protective immunity but also exert an anti-parasitic activity.

1-11. (canceled)

12. A synthetic peptide having the following sequence of amino acids (E34P) (SEQ ID No. 1): E-D-E-H-K-G-K-Y-C-R-L-G-N-D-C-R-T-T-E-P-T-T-T-A-T-P-R-G-T-P-T-P-A-P, said sequence being cyclized by means of a disulfide bridge between the cysteine at position 9 and the cysteine at position 15.

13. The synthetic peptide of claim 12, wherein said peptide induces production of IgG2 isotype specific antibodies, whereby enabling, in the case of conditions linked to a state of delayed hyperstimulation, the distinction between an infected or sick animal or human from a non-infected or healthy, but vaccinated or treated, animal or human.

14. A synthetic peptide complex, consisting of: (a) the synthetic peptide of claim 12; (b) a synthetic peptide having the following sequence (A16E) of 16 amino acids (SEQ ID No. 2) or derivatives (structural analogues) thereof: A-A-R-C-A-R-C-R-E-G-Y-S-L-T-D-E, in which C may be replaced by S and L by I; (c) a synthetic peptide having the following sequence (A16G) of 16 amino acids (SEQ ID No. 3) or derivatives (structural analogues) thereof: A-A-S-S-T-P-S-P-G-S-G-C-E-V-D-G in which C may be replaced by S and L by I; and (d) an adjuvant.

15. The synthetic peptide complex of claim 14, wherein said adjuvant induces a cell-mediated response.

16. The synthetic peptide complex of claim 14, wherein said adjuvant is QA21, saponin from quil-A, or a derivative thereof.

17. The synthetic peptide complex of claim 16, wherein said adjuvant is QS21.

18. The synthetic peptide complex of claim 14, wherein the peptide sequences E34P, A16E and A16G are provided in a weight ratio of E34P/A16E/A16G of 10/25/25.

19. The synthetic peptide complex of claim 14, wherein the complex is useful for the prevention and/or the treatment of conditions in mammals, the protective immunity of which depends on the stimulation of the Th1 type lymphocytes.

20. The synthetic peptide complex of claim 14, wherein the complex is useful for the prevention and/or the treatment of conditions in mammals, the protective immunity of which depends on a state of delayed hyperstimulation.

21. The synthetic peptide complex of claim 14, wherein any of the peptides E34P, A16E and A16 G are bound to carriers, whereby rendering the peptides immunogenic.

22. The synthetic peptide complex of claim 21, wherein the carriers are large KLH type molecules, palmitoyl-type lipopeptides or derivatives thereof.

23. The synthetic peptide complex of claim 14, wherein complex is formulated for subcutaneous, intradermal, intramuscular, intravenous, parenteral, or oral administration.

24. An in vitro diagnostic product comprising the synthetic peptide of claim claim 12.

25. The product of claim 24, wherein the product is useful for the detection of a humoral response.

26. The product of claim 24, wherein the product is useful for the detection of an IgG2 response.

27. The product of claim 24, wherein the product is useful for (a) the detection of a Th1 type immune state and of a state of delayed hyperstimulation, (b) the monitoring of the immune response in vaccinated or non-vaccinated but treated mammals, or (c) the monitoring of the efficacy of a vaccination or of chemotherapeutic and/or immunotherapeutic treatment.

28. An in vitro diagnostic product comprising the synthetic peptide complex of claim 14.

29. An in vitro diagnostic kit comprising the diagnostic product of claim 24, wherein the synthetic peptide of claim 12 is bound to a solid support.

30. An in vitro diagnostic kit comprising the diagnostic product complex of claim 28, wherein the synthetic peptide of claim 12 is bound to a solid support.

31. An in vitro diagnostic kit comprising the diagnostic product of claim 24, wherein the synthetic peptide of claim 12 is bound to nitrocellulose, a polymer membrane, latex, or plastic (polymer) material support.

32. An in vitro diagnostic kit comprising the diagnostic product complex of claim 28, wherein any of the peptides E34P, A16E and A16G are bound to nitrocellulose, a polymer membrane, latex, or plastic (polymer) material support.

33. A method for inducing in a mammal specific antibodies linked to a Th1 type immune state comprising administering to a mammal in need thereof a therapeutically effective amount of the synthetic peptide of claim 12.

34. The method of claim 33, wherein the specific antibodies are of isotype IgG2.

35. The method of claim 33, wherein the synthetic peptide of claim 12 is provided in a form of a medicine, a vaccine, an in vitro or in vivo diagnostic agent.

36. A method for inducing in a mammal specific antibodies linked to a Th1 type immune state, comprising administering to a mammal in need thereof a therapeutically effective amount of the synthetic peptide complex of claim 14.

37. The method of claim 36, wherein the specific antibodies are of isotype IgG2.

38. The method of claim 36, wherein the synthetic peptide complex of claim 14 is provided in a form of a medicine, a vaccine, an in vitro or in vivo diagnostic agent.

39. A method of preventing or treating leishmaniasis in a mammal comprising administering to a mammal in need thereof a therapeutically effective amount of the synthetic peptide of claim 12.

40. A method of preventing or treating leishmaniasis in a mammal comprising administering to a mammal in need thereof a therapeutically effective amount of the synthetic peptide complex of claim 14.

41. A method of inducing significant production of IgG2 isotype specific antibodies in a mammal comprising administering to a mammal in need thereof an effective amount of the synthetic peptide of claim 12.

42. A method of distinguishing between an infected or sick animal or human, from a non-infected or healthy, but vaccinated or treated, animal or human comprising detecting in a sample from the animal or human isotype IgG2 antibodies specific to the cyclized peptide of claim 12, wherein when isotype IgG2 antibodies are present the animal or human has been vaccinated or treated.

43. A method of neutralizing in vitro the proliferation of Leishmania sp. amastigotes and promastigotes, comprising contacting Leishmania sp. amastigotes and promastigotes with an effective amount of the synthetic peptide of claim 12.


SEQUENCE LISTING (3 sequences)

SEQ ID NO: 1 - protein, 34 aa, Artificial Sequence
  Fully-synthetic sequence
EDEHKGKYCRLGNDCRTTEPTTTATPRGTPTPAP

SEQ ID NO: 2 - protein, 16 aa, Artificial Sequence
  Fully-synthetic sequence
AARCARCREGYSLTDE

SEQ ID NO: 3 - protein, 16 aa, Artificial Sequence
  Fully-synthetic sequence
AASSTPSPGSGCEVDG